(19) United States
(12) Patent Application Publication
(10) Pub. No.: US 2010/0160621 A1
(43) Pub. Date: Jun. 24, 2010

(54) SYNTHESIS OF ALKOXYLATED SUCROSE ESTERS

(76) Inventor: Jared John Schaefer, Wyoming, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/637,040

(22) Filed: Dec. 14, 2009

Related U.S. Application Data
(63) Continuation of application No. 11/158,355, filed on Jun. 20, 2005, now abandoned.
(60) Provisional application No. 60/606,074, filed on Aug. 31, 2004.

(51) Int. Cl. C07H 3/04

(52) U.S. Cl. 536/115

(57) ABSTRACT

A process for the preparation of an alkoxylated sucrose ester including the steps of:
forming an initial reaction mixture of a sucrose ester and from about 0.01% to about 5% of a catalyst; and forming an initial reaction product by reacting the initial reaction mixture with an epoxide for a period of time in the range of from about 30 minutes to about 6 hours and at a temperature in the range of from about 80° C. to about 120° C.; wherein the epoxide and the sucrose ester are selected such that a mole ratio of epoxide groups to sucrose hydroxyls is from about 1 to about 100. Useful catalysts include sodium metals, potassium metals, sodium/potassium alloys, and mixtures thereof

### CROSS REFERENCE TO RELATED APPLICATIONS

This application is a continuation of and claims priority under 35 U.S.C. §120 to U.S. patent application Ser. No. 11/158,355, filed Jun. 20, 2005, which in turn claims the benefit of priority to U.S. Provisional Application Ser. No. 60/606,074, filed Aug. 31, 2004, which is herein incorporated by reference.

### BRIEF DESCRIPTION OF THE INVENTION

The present invention relates to the improved production of alkoxylated sucrose esters. Such improved production may be carried out without a solvent.

### BACKGROUND OF THE INVENTION

There currently exists several methods for producing sucrose esters of varying degrees of esterification. For instance, Rizzi and Taylor, U.S. Pat. No. 3,963,699, describe a solvent-free transesterification process in which a mixture of a polyol (such as sucrose), a fatty acid lower alkyl ester (such as a fatty acid methyl ester), an alkali metal fatty acid soap, and a basic catalyst is heated to form a homogenous melt, to which is added excess fatty acid lower alkyl ester to form the higher polyol fatty acid polyesters.

Feuge et al, U.S. Pat. No. 3,714,144, and Feuge et al, J. Amer. Oil Chem. Soc., 1970, 47(2), 56-60, disclose a solvent-free transesterification process which comprises mixing molten sucrose with esters of fatty acids and alkali-free sodium or potassium soaps under a partial vacuum. The teachings of Feuge et al include the formation of lower esters; the only specific teaching by Feuge et al. of a method in which the percentage of sucrose esters having three or more fatty acid chains is greater than 35% of the total sucrose esters formed uses methyl carbitol palmitate as a fatty acid source.

Osipow et al, U.S. Pat. No. 4,380,616, disclose the preparation of sucrose mono- and di-esters by forming a transparent emulsion containing immiscible reactants and maintaining the transparent emulsions under appropriate conditions to permit reaction. Sucrose mono- and di-esters are formed using emulsions containing methyl fatty acid ester and sucrose. Osipow et al. also disclose the formation of mono- and di-glycerides using emulsions containing glycerine and methyl fatty acid esters or glycerol tri-esters.

There also currently exists several methods for producing alkoxylated sucrose esters. Ennis et al, U.S. Pat. No. 5,077,073, disclose a process for preparing alkoxylated sucrose esters made from alkoxylated sucrose that is then reacted in a solvent to form the alkoxylated sucrose esters. This material is then used as a fat substitute. Ferenz, U.S. Pat. No. 5,427,815, discloses a process for preparing linked, alkoxylated, esterified polyols made from alkoxylated polyol that is then esterified with a polycarboxylate segment. Porta et al., U.S. Pat. No. 6,486,120, disclose the use of alkoxylated sucrose esters in liquid, aqueous softening compositions. Again, the methodology disclosed involves first alkoxylating the sucrose in a solvent and then esterifying to form the alkoxylated sucrose esters. Cooper, U.S. Pat. No. 5,118,448, discloses a process for preparing alkoxylated esterified polyols that involves first forming a benzylated polyol that is then alkoxylated. The benzylated, alkoxylated polyol is then converted to an alkoxylated polyol and then esterified. This process also involves alkoxylating prior to esterifying, though it claims to produce a material with at least one ester group directly bonded to the polyol backbone.

These known inventions for making alkoxylated sucrose esters may have disadvantages. First, they may suffer from the necessity of using a solvent to first alkoxylate the sucrose. Such solvent use may be expensive and require additional processing steps for obtaining a purified resulting product.

Secondly, in known process, where the sucrose is first alkoxylated and then esterified, all of the ester groups reside some distance away from the sucrose molecule based on the number of alkoxyl groups reacted with the hydroxyls of the sucrose, as the hydroxyl sites for esterification are moved away from the molecule by the alkoxyl groups.

Therefore, a need exists for an improved process for producing alkoxylated sucrose esters having at least some of the ester groups residing near the sucrose molecule. Furthermore, a need exists for such a process wherein the process may be solvent-free.

### SUMMARY OF THE INVENTION

The present invention relates to processes for the preparation of an alkoxylated sucrose ester, wherein the process comprises the steps of: a) forming an initial reaction mixture, wherein said initial reaction mixture comprises: a sucrose ester and from about 0.01% to about 5%, by weight of the initial reaction mixture, of a catalyst; and b) forming an initial reaction product by reacting the initial reaction mixture with an epoxide for a period of time in the range of from about 30 minutes to about 6 hours and at a temperature in the range of from about 80° C. to about 120° C.; wherein the epoxide and the sucrose ester are selected such that a mole ratio of epoxide groups to sucrose hydroxyls is from about 1 to about 100.

In one embodiment, the initial reaction mixture further comprises less than about 5%, by weight of the initial reaction mixture, of a solvent.

In one embodiment, initial reaction mixture is substantially free of solvent.

In one embodiment, the sucrose ester is selected from sucrose esters or mixtures of sucrose esters having an average degree of esterification of from about 1% to about 99%.

In one embodiment, the catalyst is selected from sodium metals, potassium metals, sodium/potassium alloys, and mixtures thereof.

In one embodiment, the epoxide is selected from ethylene oxide, propylene oxide, butylene oxide, and mixtures thereof.

In one embodiment, the initial reaction mixture comprises less than about 5% of a solvent selected from dimethyl formamide, dimethyl sulfoxide, or mixtures thereof.

In one embodiment, the initial reaction product is formed in an atmosphere containing the epoxide and an inert gas.

In one embodiment, the process comprises the steps of: forming an initial reaction mixture, wherein said initial reaction mixture comprises: i) from about 0.99% to about 99.99% of a sucrose ester; and ii) from about 0.01% to about 5% of a catalyst;

and then forming an initial reaction product by reacting the initial reaction mixture with an epoxide for a period of time in the range of from about 30 minutes to about 6 hours, and at a temperature in the range of from about 80° C. to about 120° C.; then c) forming a purified reaction product by washing said initial reaction product with an aqueous washing solution at a temperature of from about 20° C. to about 100° C., gently stirring, and allowing the resulting two phases to separate; and then d) isolating the impurities from said purified reaction product; wherein the ratio of the epoxide to the sucrose ester is selected such that a mole ratio of epoxide groups per sucrose hydroxyls is from about 1 to about 50.

In one embodiment, the aqueous water washing solution is added in amount that is from about 1% to about 50%, by weight of the reaction product.

In one embodiment, the process comprises the steps of: forming an initial reaction mixture, wherein said initial reaction mixture comprises: i) a sucrose ester; ii) from about 0.01% to about 5% of a catalyst selected from sodium metals, potassium metals, sodium/potassium alloys, and mixtures thereof; then forming an initial reaction product by reacting the initial reaction mixture in an atmosphere for a period of time in the range of from about 30 minutes to about 6 hours, and at a temperature in the range of from about 80° C. to about 120° C., wherein the atmosphere comprises: i) from about 0.1% to about 100%, by volume of the atmosphere of the epoxide; and ii) an inert gas; and then forming a purified reaction product by sparging the initial reaction product with nitrogen for a time period of from about 1 minute to about 2 hours at a temperature of from about 20° C. to about 100° C.; wherein the ratio of the epoxide to the sucrose ester is selected such that a mole ratio of epoxide groups per sucrose hydroxyls is from about 1 to about 100.

The present invention also relates to alkoxylated sucrose esters formed by the processes described herein.

### DETAILED DESCRIPTION OF THE INVENTION

The present invention encompasses processes for the preparation of alkoxylated sucrose esters having at least some of the ester groups residing near the sucrose molecule. The present invention will now be described in detail with reference to specific embodiments.

### A. Definitions

Various publications and patents are referenced throughout this disclosure. All references cited herein are hereby incorporated by reference. Unless otherwise indicated, all percentages and ratios are calculated by weight. All percentages and ratios are calculated based on the total dry composition unless otherwise indicated.

All component or composition levels are in reference to the active level of that component or composition, and are exclusive of impurities, for example, residual solvents or by-products, which may be present in commercially available sources.

Referred to herein are trade names for components including various ingredients utilized in the present invention. The inventors herein do not intend to be limited by materials under a certain trade name. Equivalent materials (e.g., those obtained from a different source under a different name or catalog number) to those referenced by trade name may be substituted and utilized in the compositions, kits, and methods herein.

As used herein, and unless otherwise indicated, the use of a numeric range to indicate the value of a given variable is not intended to be limited to just discrete points within that stated range. One of ordinary skill in the art will appreciate that the use of a numeric range to indicate the value of a variable is meant to include not just the values bounding the stated range, but also all values and sub-ranges contained therein. By way of example, consider variable X that is disclosed as having a value in the range of A to B. One of ordinary skill in the art will understand that variable X is meant to include all integer and non-integer values bounded by the stated range of A to B. Moreover, one of ordinary skill in the art will appreciate that the value of the variable also includes all combinations and/or permutations of sub-ranges bounded by the integer and non-integer values within and including A and B.

As used herein, the term “degree of esterification” refers to the average percentage of hydroxyl groups of a polyol composition that have been esterified.

In one embodiment of the present invention the polyol is sucrose having eight hydroxyl groups and has a degree of esterification of from about 30% to about 90%. As used herein the degree of esterification calculation does not include non-esterified polyol compounds that may be present.

In the description of the invention various embodiments and/or individual features are disclosed. As will be apparent to the ordinarily skilled practitioner, all combinations of such embodiments and features are possible and can result in preferred executions of the present invention.

### B. Processes for Preparing Alkoxylated Sucrose Ester Compositions

Sucrose esters are defined as sucrose molecules that have been esterified with between, on average, one to eight ester groups on the eight available hydroxyls. Depending on their degree of esterification, sucrose esters can either be solids or liquids. These sucrose esters or mixtures thereof can then be alkoxylated by reacting them with epoxide or mixtures thereof, which involves a reaction at the non-esterified hydroxyl sites. This reaction may utilize a solvent if the starting sucrose ester is a solid or a liquid, or alternately, does not require a solvent when the starting sucrose esters or mixtures thereof are a liquid.

The improved processes of the present invention involve alkoxylating sucrose esters or mixtures thereof that have already been esterified to a varying degree using any of the commonly known processes for sucrose esterification. Without being limited by theory, it is believed that these alkoxylated sucrose esters exhibit interesting properties as surfactants, lubricants, and cleaning agents, and exhibit different properties from either the starting sucrose esters or those alkoxylated sucrose esters formed by the previously known processes of first alkoxylating sucrose and then esterifying.

It has now been surprisingly found that the improved processes of the present invention produce alkoxylated sucrose esters that are different in composition from those alkoxylated sucrose esters formed when sucrose is first alkoxylated and then esterified. Without being limited by theory, when the sucrose is first esterified and then alkoxylated, it is believed that at least some of the ester groups now exist on the sucrose molecule itself, and may or may not move to locations farther away from the sucrose as the alkoxylation progresses. It is believed that, for this reason, the composition and performance of the alkoxylated sucrose esters made using the improved processes herein are different from that exhibited by the alkoxylated sucrose esters prepared using processes previously known.

The chemical structures of the composition resulting from the processes disclosed herein and that which results from the prior art can be illustrated by the general structure shown below. For illustration purposes, the molecule in the general structure shown below is assumed to have been reacted with ethylene oxide, a common epoxide. This is not intended to limit the scope of the disclosed composition to this one type of alkoxylated sucrose ester. The general structure shown below is intended to represent both the composition that results when the prior art or the disclosed process is utilized, and the differences described in the previous paragraph are highlighted by the discussion below.

### General Structure of Alkoxylated Sucrose Ester

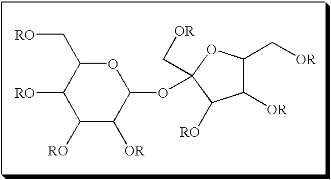

where R is independently selected from:
i) COR′ (ester group);
ii) (CH₂CH₂O)ₓH (ethoxide group, where x=1−50);
iii) ((CH₂CH₂O)ₓ COR′ (ethoxide group that has been esterified, where x=1-50); and
iv) mixtures thereof.

Without being limited by theory, it is now believed that the number of ester groups, ethoxide groups, and esterified ethoxide groups on the sucrose depends on the order in which the esterification and ethoxylation are carried out. In the case of the previously known processes where the sucrose was first ethoxylated and then esterified, the resulting molecules have ester groups existing as esters on the end of a number of ethoxide groups (i.e., structure iii). These materials will not have any ester groups directly esterified to the sucrose backbone (structure i), as the sites for esterification have been moved away from the sucrose backbone during the previous ethoxylation step. In contrast, the alkoxylated sucrose esters resulting from the novel processes disclosed herein, will have a finite number of ester groups that exist directly bonded to the sucrose backbone, as the starting raw material is a sucrose ester, which by definition consists of ester groups directly esterified to the hydroxyls of sucrose. By the processes herein, these sucrose esters are then ethoxylated, which produces ethoxide groups as shown by structure ii.

Therefore, the present invention encompasses alkoxylation processes for the production of alkoxylated sucrose esters. The present invention will now be described in detail with reference to specific embodiments.

In general, the processes for the preparation of alkoxylate sucrose esters of the present invention include the steps of forming an initial reaction product from an initial reaction mixture; optionally washing the reaction product to remove impurities; optionally sparging the reaction product with nitrogen; optionally subjecting the reaction product to a vacuum; and optionally drying a purified reaction product. Preferably, no reaction solvent is used during the preparation process so that there is no reaction solvent residual to be removed.

Initial Reaction Mixture

An initial reaction mixture is formed by adding sucrose esters, or mixtures thereof, to a suitable reaction vessel. The initial reaction mixture contains a sucrose ester and a catalyst. In one embodiment, the initial reaction mixture contains a solvent.

In one embodiment, the initial reaction mixture contains from about 0.1% to about 99.99%, by weight of the initial reaction mixture, of the sucrose ester. Preferably, the initial reaction contains from about 90% to about 99.9%, by weight of the initial reaction mixture, of the sucrose ester, alternatively from about 95% to about 99%, still alternatively from about 97% to about 99%. Suitable sucrose esters for use herein include those having an average degree of esterification of from about 1% to about 99%, preferably having a degree of esterification of from about 25% to about 90%, alternatively from about 30% to about 80%. Sucrose esters useful herein include sucrose mono, di, tri, tetra, penta, hexa, and heptaesters.

In one embodiment, the initial reaction mixture comprises from about 0.01% to about 99%, by weight of the initial reaction mixture, of an alkoxylation catalyst. Preferably, the initial reaction mixture comprises from about 1% to about 10%, by weight of the initial reaction mixture, of the catalyst, alternatively from about 2% to about 5%. Suitable catalysts for use herein include sodium metals, potassium metals, sodium/potassium alloys, and mixtures thereof.

In one embodiment, the initial reaction mixture contains a solvent. Optionally, a solvent may be used (although not preferred) if the sucrose esters or mixtures thereof do not form a liquid reaction medium. When present, the initial reaction mixture may comprise from about 0.01% to about 99.89% of a solvent. Preferably, the initial reaction mixture comprises less than 5% of solvent, alternatively less than about 1% of solvent, alternatively is substantially free of solvent. As used herein, “substantially free of solvent” refers to a composition which comprises no readily detectable level of solvent. When included, solvents that may be used herein include materials such as dimethyl sulfoxide and/or dimethyl formamide.

The reaction vessel is set up such that the epoxide or mixtures thereof can also be added at the time the initial reaction product is to be made.

Initial Reaction Product

The alkoxylated sucrose esters of the present invention are formed by first forming an initial reaction product. The initial reaction product is formed by reacting the initial reaction mixture with an epoxide. The initial reaction mixture and the epoxide are preferably reacted for a period of time in the range of from about 30 minutes to about 6 hours and at a temperature in the range of from about 80° C. to about 120° C.

The ratio of the epoxide to the sucrose ester is selected such that the mole ratio of epoxide groups to sucrose hydroxyls is from about 1 to about 100, alternatively from about 1 to about 50; alternatively from about 1 to about 30; alternatively from about 1 to about 20.

Typically, initial reaction product is formed by reacting the initial reaction mixture in a vessel containing an atmosphere that includes the epoxide and may include an inert gas, such as nitrogen. Epoxides suitable for use herein include ethylene oxide, propylene oxide, butylene oxide, and mixtures thereof. In one embodiment, the initial reaction product is formed while the epoxide is injected into the atmosphere around the initial reaction mixture. In another embodiment, the initial reaction product is formed in a reactor wherein the epoxide is injected into the atmosphere of the reactor. In one embodiment, the epoxide is added to the atmosphere of the initial reaction mixture in a continuous feed until the requisite amount of epoxide has been added.

In one embodiment, the initial reaction mixture is reacted in the atmosphere for a period of time in the range of from about 10 minutes to about 12 hours, and at a temperature in the range of from about 80° C. to about 120° C. The atmosphere within the reactor can be in the range from about 0.1% to 100% of the epoxide, or alternately, may also contain from about 0% to 99.9% of an inert gas such as nitrogen. The epoxide or mixtures thereof is reacted with the sucrose esters or mixtures thereof until the desired amount of alkoxyl groups has been added to the hydroxyl sites of the sucrose ester.

Purification

Optionally, the alkoxyated sucrose esters may be purified by adding between about 1% to about 50% by weight of the initial reaction product, of water and/or alcohol at a temperature between about 20° C. and 100° C., gently stifling, and allowing the two phases to separate. The aqueous or alcohol phase can then be removed by traditional separation means, the impurities isolated from the purified reaction product and the purified alkoxylated sucrose ester phase is retained. Suitable alcohols for the purification include methanol, ethanol, propanol, and butanol. Alternately, if this water or alcohol washing step does not remove the desired impurities or if it is undesirable to add water or alcohol, the alkoxylated sucrose esters may be sparged with an inert gas such as nitrogen and/or subjected to a vacuum to remove any unreacted epoxide.

### EXAMPLES

The following are non-limiting examples of alkoxylated sucrose ester preparation processes, in accordance with the present invention. The following examples are provided to illustrate the invention and are not intended to limit the spirit or scope thereof in any manner.

### Example 1

Approximately 100 g of sucrose esters with an average degree of esterification of about 5 are prepared according to Rizzi and Taylor, U.S. Pat. No. 3,963,699. The sucrose esters are then placed in a reactor, and 1 g of sodium/potassium alloy is added to the reactor. The mixture is heated to 100° C. and 32 g of ethylene oxide is gradually fed into the reactor to maintain the system pressure to about 50 psi. The reaction is allowed to proceed for about 2 hours, or until all 32 g of the ethylene oxide is reacted with the sucrose esters and then the ethylene oxide feed is stopped. The initial reaction product is then cooled to 60° C. The initial reaction product now weighs approximately 132 g, which corresponds to an addition of, on average, 4 ethylene oxide groups on each of the three available hydroxyl groups.

The initial reaction product is then washed with 24 g of water at 60° C. The water and initial reaction product are gently stirred for approximately 10 minutes, and the resulting mixture is centrifuged. The top phase is decanted and retained and the bottom, aqueous phase is discarded.

The purified alkoxylated sucrose ester is analyzed and contains greater than 99.9% alkoxylated sucrose ester; less than 0.01% aldehyde; less than 0.01% ketones; less than 0.01% benzyl halide; less than 0.01% mono-benzyl ether; less than 0.01% acetals; and less than 0.01% ketals.

### Example 2

Approximately 100 g of sucrose esters with an average degree of esterification of about 4 are prepared according to Rizzi and Taylor, U.S. Pat. No. 3,963,699. The sucrose esters are then placed in a reactor, and 1 g of sodium/potassium alloy is added to the reactor. The mixture is heated to 100° C. and 125 g of ethylene oxide is gradually fed into the reactor to maintain the system pressure to about 50 psi. The reaction is allowed to proceed for about 4 hours, or until the 125 g of ethylene oxide is reacted with the sucrose esters, and then the ethylene oxide feed is stopped. The initial reaction product is then cooled to 60° C. The initial reaction product now weighs approximately 225 g, which corresponds to an addition of, on average, 10 ethylene oxide groups on each of the four available hydroxyl groups.

The initial reaction product is then washed with 50 g of water at 60° C. The water and initial reaction product are gently stirred for approximately 10 minutes, and the resulting mixture is centrifuged. The top phase is decanted and retained and the bottom, aqueous phase is discarded.

The purified alkoxylated sucrose ester is analyzed and contains: greater than 99.9% Alkoxylated Sucrose Ester; less than 0.01% aldehyde; less than 0.01% ketones; less than 0.01% benzyl halide; less than 0.01% mono-benzyl ether; less than 0.01% acetals; and less than 0.01% ketals.

### Example 3

Approximately 100 g of sucrose esters with an average degree of esterification of about 6 are prepared according to Rizzi and Taylor, U.S. Pat. No. 3,963,699. The sucrose esters are then placed in a reactor, and 1 g of sodium/potassium alloy is added to the reactor. The mixture is heated to 110° C. and 45 g of ethylene oxide is gradually fed into the reactor to maintain the system pressure to about 50 psi. The reaction is allowed to proceed for about 4 hours, or until the 45 g of ethylene oxide is reacted with the sucrose esters, and then the ethylene oxide feed is stopped. The initial reaction product is then cooled to 60° C. The initial reaction product now weighs approximately 145 g, which corresponds to an addition of, on average, 10 ethylene oxide groups on each of the two available hydroxyl groups.

The initial reaction product is then washed with 40 g of ethanol at 60° C. The ethanol and initial reaction product are gently stirred for approximately 10 minutes, and the resulting mixture is centrifuged. The top phase is decanted and retained and the bottom, alcohol phase is discarded.

The alkoxylated sucrose ester product is then analyzed and contains less than 1% aldehydes; less than 1% ketones; less than 1% benzyl halide; less than 1% mono-benzyl ether; less than 1% acetals; and less than 1% ketals.

### Example 4

Approximately 50 g of sucrose esters with an average degree of esterification of about 4 and approximately 50 g of sucrose esters with an average degree of esterification of about 7 are prepared according to Rizzi and Taylor, U.S. Pat. No. 3,963,699. The sucrose esters are then combined and then placed in a reactor, and 1 g of sodium/potassium alloy is added to the reactor. The mixture is heated to 90° C. and 60 g of ethylene oxide is gradually fed into the reactor to maintain the system pressure to about 50 psi. The reaction is allowed to proceed for about 4 hours, or until the 60 g of ethylene oxide is reacted with the sucrose esters, and then the ethylene oxide feed is stopped. The initial reaction product is then cooled to 60° C. The initial reaction product now weighs approximately 160 g.

The initial reaction product is then washed with 30 g of water at 60° C. The water and initial reaction product are gently stirred for approximately 10 minutes, and the resulting mixture is centrifuged. The top phase is decanted and retained and the bottom, aqueous phase is discarded.

The alkoxylated sucrose ester product is then analyzed and contains less than 1% aldehydes; less than 1% ketones; less than 1% benzyl halide; less than 1% mono-benzyl ether; less than 1% acetals; and less than 1% ketals.

### Example 5

Approximately 100 g of sucrose esters with an average degree of esterification of about 5 are prepared according to Rizzi and Taylor, U.S. Pat. No. 3,963,699. The sucrose esters are then placed in a reactor, and 1 g of sodium/potassium alloy is added to the reactor. The mixture is heated to 100° C. and 63 g propylene oxide is gradually fed into the reactor to maintain the system pressure to about 50 psi. The reaction is allowed to proceed for about 2 hours, or until the 83 g propylene oxide is reacted with the sucrose esters, and then the propylene oxide feed is stopped. The initial reaction product is then cooled to 60° C. The initial reaction product now weighs approximately 163 g, which corresponds to an addition of, on average, 8 propylene oxide groups on each of the three available hydroxyl groups.

The initial reaction product is then washed with 40 g of water at 60° C. The water and initial reaction product are gently stirred for approximately 10 minutes, and the resulting mixture is centrifuged. The top phase is decanted and retained and the bottom, aqueous phase is discarded.

The alkoxylated sucrose ester product is then analyzed and contains less than 1% aldehydes; less than 1% ketones; less than 1% benzyl halide; less than 1% mono-benzyl ether; less than 1% acetals; and less than 1% ketals.

### Example 6

Approximately 100 g of sucrose esters with an average degree of esterification of about 2 are prepared according to Osipow et al, U.S. Pat. No. 4,380,616. The sucrose esters are dissolved in 300 g of dimethyl sulfoxide and the mixture is then placed in a reactor. To this is added 2 g of sodium/potassium alloy. The mixture is heated to 90° C. and 300 g ethylene oxide is gradually fed into the reactor to maintain the system pressure at about 50 psi. The reaction is allowed to proceed for 2 hours, or until the 300 g ethylene oxide is reacted with the sucrose esters, and then the ethylene oxide feed is stopped. The initial reaction product is then cooled to 60° C. The initial reaction product, including the solvent, now weighs approximately 700 g, which corresponds to an addition of, on average, 10 ethylene oxide groups on each of the six available hydroxyl groups.

The initial reaction product is then purified by sparging with nitrogen at 60° C. for 1 hour.

The alkoxylated sucrose ester product is then analyzed and contains less than 1% aldehydes; less than 1% ketones; less than 1% benzyl halide; less than 1% mono-benzyl ether; less than 1% acetals; and less than 1% ketals.

What is claimed is:

1. A process for the preparation of an alkoxylated sucrose ester, wherein the process comprises the steps of:
a) forming an initial reaction mixture, wherein said initial reaction mixture comprises:
i) a sucrose ester; and
ii) from about 0.01% to about 5%, by weight of the initial reaction mixture, of a catalyst; and
b) forming an initial reaction product by reacting the initial reaction mixture with an epoxide for a period of time in the range of from about 30 minutes to about 6 hours and at a temperature in the range of from about 80° C. to about 120° C.;
wherein the epoxide and the sucrose ester are selected such that a mole ratio of epoxide groups to sucrose hydroxyls is from about 1 to about 100.

2. A process according to claim 1 wherein the initial reaction mixture further comprises less than about 5%, by weight of the initial reaction mixture, of a solvent.

3. A process according to claim 1 wherein the initial reaction mixture is substantially free of solvent.

4. A process according to claim 1 wherein the sucrose ester is selected from sucrose esters or mixtures of sucrose esters having an average degree of esterification of from about 1% to about 99%.

5. A process according to claim 4 wherein the catalyst is selected from sodium metals, potassium metals, sodium/potassium alloys, and mixtures thereof.

6. A process according to claim 4 wherein said epoxide is selected from ethylene oxide, propylene oxide, butylene oxide, and mixtures thereof.

7. A process according to claim 4 wherein the initial reaction mixture comprises less than about 5% of a solvent selected from dimethyl formamide, dimethyl sulfoxide, or mixtures thereof.

8. A process according to claim 1 wherein the initial reaction product is formed in an atmosphere, said atmosphere consisting essentially of the epoxide and an inert gas.

9. A process for the preparation of an alkoxylated sucrose ester, wherein the process comprises the steps of:
a) forming an initial reaction mixture, wherein said initial reaction mixture comprises:
i) from about 0.99% to about 99.99% of a sucrose ester; and
ii) from about 0.01% to about 5% of a catalyst; and
b) forming an initial reaction product by reacting the initial reaction mixture with an epoxide for a period of time in the range of from about 30 minutes to about 6 hours, and at a temperature in the range of from about 80° C. to about 120° C.;
c) forming a purified reaction product by washing said initial reaction product with an aqueous washing solution at a temperature of from about 20° C. to about 100° C., gently stifling, and allowing the resulting two phases to separate; and then
d) isolating the impurities from said purified reaction product;
wherein the ratio of the epoxide to the sucrose ester is selected such that a mole ratio of epoxide groups per sucrose hydroxyls is from about 1 to about 50.

10. A process according to claim 9 wherein the aqueous water washing solution is added in amount that is from about 1% to about 50%, by weight of the reaction product.

11. A process according to claim 9 wherein the initial reaction mixture further comprises less than about 5% of a solvent.

12. A process according to claim 9 wherein the initial reaction mixture is substantially free of solvent.

13. A process according to claim 9 wherein the sucrose ester is selected from sucrose esters or mixtures of sucrose esters having an average degree of esterification of from about 1% to about 99%.

14. A process according to claim 13 wherein the catalyst is selected from sodium metals, potassium metals, sodium/potassium alloys, and mixtures thereof.

15. A process according to claim 11 wherein said epoxide is selected from ethylene oxide, propylene oxide, butylene oxide, and mixtures thereof.

16. A process according to claim 11 wherein the initial reaction mixture comprises less than 5% of a solvent selected from dimethyl formamide, dimethyl sulfoxide, and mixtures thereof.

17. A process according to claim 9 wherein the initial reaction product is formed in an atmosphere, said atmosphere consisting essentially of the epoxide and nitrogen.

18. A process for the preparation of an alkoxylated sucrose ester, wherein the process comprises the steps of:
a) forming an initial reaction mixture, wherein said initial reaction mixture comprises:
i) a sucrose ester;
ii) from about 0.01% to about 5% of a catalyst selected from sodium metals, potassium metals, sodium/potassium alloys, and mixtures thereof,
b) forming an initial reaction product by reacting the initial reaction mixture in an atmosphere for a period of time in the range of from about 30 minutes to about 6 hours, and at a temperature in the range of from about 80° C. to about 120° C., wherein the atmosphere comprises:
i) from about 0.1% to about 100%, by volume of the atmosphere of the epoxide; and
ii) an inert gas; and
c) forming a purified reaction product by sparging the initial reaction product with nitrogen for a time period of from about 1 minute to about 2 hours at a temperature of from about 20° C. to about 100° C.;
wherein the ratio of the epoxide to the sucrose ester is selected such that a mole ratio of epoxide groups per sucrose hydroxyls is from about 1 to about 100.

19. A process according to claim 1 wherein the process further comprises the step of:
c) forming a purified reaction product by subjecting the initial reaction product to a vacuum for a time period of from about 1 minute to about 2 hours at a temperature of from about 20° C. to about 100° C.

20. An alkoxylated sucrose ester formed by the process according to claim 1.
